(19) United States
(12) Patent Application Publication
(10) Pub. No.: US 2023/0172189 A1
(43) Pub. Date: Jun. 8, 2023

(54) Methods for Extending the Shelf-life of Stored Donor Blood and/or Red Blood Cells and Treated Red Blood Cell Compositions Produced Thereby

(71) Applicant: Ohio University, Athens, OH (US)

(72) Inventors: Amir M. Farnoud, Athens, OH (US); Amid Vahedi, Athens, OH (US)

(73) Assignee: Ohio University, Athens, OH (US)

(21) Appl. No.: 17/919,364

(22) PCT Filed: Apr. 26, 2021
(86) PCT No.: PCT/US2021/029104

§ 371 Date: Oct. 17, 2022

Related U.S. Application Data
(60) Provisional application No. 63/016,395, filed on Apr. 28, 2020.

(51) Int. Cl. A01N 1/02; C12N 5/078

(52) U.S. Cl. CPC A01N 1/0226; C12N 5/0641

(57) ABSTRACT

Methods for extending the shelf life of donor blood and/or red blood cells (RBC) with a cyclodextrin composition are described. The cyclodextrin composition contains at least one cyclodextrin complexed with at least one exogenous lipid. Also described are such treated donor blood and/or RBC.

### RELATED APPLICATIONS

This application claims priority to U.S. Provisional Application Ser. No. 63/016,395 filed under 35 U.S.C. § 111(b) on Apr. 28, 2020, the disclosure of which is incorporated herein by reference in its entirety.

### STATEMENT REGARDING FEDERALLY SPONSORED RESEARCH

This invention was made with government support under Grant No. 1903568 awarded by the National Science Foundation. The government has certain rights in the invention.

### FIELD OF THE INVENTION

This invention relates to a method for enhancing the shelf life of donor blood. More particularly, the invention relates to a method for enhancing the shelf life of red blood cells.

### BACKGROUND OF THE INVENTION

The need for blood and blood products is always great, and there is a continuing large need for donor blood. Therefore, numerous blood banks and donor programs have been established by the Red Cross and other organizations. One problem associated with donor blood, however, is its short shelf life. Red blood cells, the most frequently used component, has a shelf life of 35 to 42 days at refrigerated temperatures. This limited shelf life makes it difficult to keep a readily available supply of red blood cell.

After 42 days, many red blood cells (RBCs) become senescent and are phagocytized upon transfusion into a recipient. In storage, RBCs generally undergo progressive degradation of the cells over time, which results—when transfused into a recipient—in physical properties that enhance the rate of clearance from the circulation.

Also, as stored blood ages, the chances of morbidity resulting from a transfusion increases. Morbidity occurs due to significant uptake of aged RBCs by the monocyte/macrophage system, resulting in significant iron deposition in the tissues, which leads to inflammation.

RBCs are most often stored before transfusion, there is a limit to the availability of transfusable blood, and expired blood is no longer viable for transfusion.

As such there are urgent economic and medical needs to extend the shelf life of stored RBCs.

There is also a need for a method to be able to rejuvenate already stored RBCs which are functioning sub-optimally.

As such, there is a great need for methods to extend the useful life of RBCs.

There is no admission that the background art disclosed in this section legally constitutes prior art.

### SUMMARY OF THE INVENTION

In a first broad aspect, described herein is a method for extending the shelf life of donor blood containing red blood cells (RBC), comprising:

i) obtaining, or having obtained, a supply of donor blood and/or RBC from a subject;

ii) exposing the donor blood and/or RBC to a quantity of a cyclodextrin composition, wherein the cyclodextrin composition comprises at least one cyclodextrin complexed with at least one exogenous lipid, in an amount sufficient to mediate the exchange of the exogenous lipids with at least one type of lipid present on an outer membrane of the RBC in the donor blood; and,

iii) extending the shelf life of the donor blood and/or RBC.

In one embodiment, the method further includes the step of preserving the cyclodextrin-treated donor blood and/or RBC of step iii) in storage.

In one embodiment, the cyclodextrin comprises methyl-α-cyclodextrin (MαCD).

In one embodiment, the exogenous lipids comprise one or more of: phosphatidylcholines (PC) and sphingomyelins (SM).

In one embodiment, the donor blood and/or RBC are exposed to the cyclodextrin composition for a period of time sufficient to reconstitute the lipids in the outer leaflet of the membranes of the RBC in the donor blood.

In one embodiment, the amount of time of cyclodextrin composition exposure ranges from about 15 minutes to about 2 hours, from about 30 minutes to about hour, or about 1 hour.

In one embodiment, MαCD-treated RBCs have a shelf life of 42 or more, for up to 210 days.

In one embodiment, the blood is mammalian blood, and/or the RBC are from mammalian blood.

In one embodiment, the blood is human blood, and/or the RBC are from human blood.

In one embodiment, MαCD is added at a concentration of between 10 mM and 20 mM of cyclodextrin composition.

In one embodiment, the mediation is determined by measuring the membrane integrity of the cyclodextrin composition-treated RBC as compared to non-cyclodextrin composition-treated RBC.

In another aspect, also described herein is a kit intended for the treatment of red blood cells (RBC) in donor blood, the kit comprising: a quantity of a cyclodextrin composition described herein, and instructions for treating the RBC in donor blood.

In one embodiment, the kit further includes the cyclodextrin at a determined concentration, complexed with lipids.

In another aspect, also described herein is a unit of red blood cells suitable for infusion into a subject comprising a supply of cyclodextrin-composition-treated red blood cells.

In another aspect, also described herein is a method of infusing red blood cells into a subject in need thereof, comprising infusing into the subject a unit of cyclodextrin-composition-treated RBC.

In another aspect, also described herein is a red blood cell composition comprising cyclodextrin-composition-treated RBC, and optionally, one or more of anticoagulants, pathogen-inactivating compounds, and other red blood cell additive solutions.

In another aspect, also described herein is a method for decreasing morbidity of a blood transfusion recipient, comprising: treating the blood transfusion recipient with an effective amount of a cyclodextrin-treated red blood cells (RBC) having a quantity of RBC having a quantity of exogenous lipids present on the outer membranes of the RBC, wherein the exogenous lipids are complexed with a cyclodextrin; and, decreasing the morbidity of the blood transfusion recipient

Various objects and advantages of this invention will become apparent to those skilled in the art from the following detailed description of the preferred embodiment, when read in light of the accompanying drawings.

### BRIEF DESCRIPTION OF THE DRAWING

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIGS. 1A-1B: The in vitro clearance of senescent red blood cells before and after exchange with 1:1 bSM:POPC:

FIG. 1A: Flow cytometry results of senescent red blood cells before and after lipid exchange. The y-axis depicts the fluorescence intensity of FITC, conjugated to lactadherin, a known PS binding molecule. The red curve represents untreated cells and the blue curve represents the cells with exchanged outer leaflet.

FIG. 1B: In vitro macrophage uptake of senescent red blood cells before and after exchange. Results are presented as mean±standard deviation of 3 independent experiments.

FIGS. 2A-2C: Confocal microscopy images of RBCs. The fluorescence of NBD is shown in green:

FIG. 2A: Cells incubated with 1:19 NBD-PE:POPS molar ratio in the absence of MαCD.

FIG. 2B: Cells incubated with 1:19 NBD-PE:POPS molar ratio in the presence of MαCD.

FIG. 2C: Cells incubated with 1:19 NBD-PE:POPS molar ratio in the presence of MαCD and treated with 50 mM of sodium dithionite, to quench the NBD fluorescence, immediately before imaging,

FIG. 3: Thin layer chromatography of the lipids in the supernatant of the lipid exchange process. The concentration of MαCD was 20 mM. The loaded lipids are present in excess in the supernatant of the exchange process, causing large dark bands on the TLC plate. In lanes 1 and 2, the loaded lipid was bSM, in lines 3 and 4 the loaded lipid was POPC and in lanes 5 and 6 the loaded lipid was POPS. The concentration of loaded lipids in the odd numbered lanes is twice the concentration of loaded lipids in the even numbered lanes. Lane 7 shows bands for total phospholipid lipid extraction of RBCs.

FIGS. 4A-4E: Abundance of phospholipids and their species in the RBCs before and after exchange with POPS:

FIG. 4A: Molar percentage abundance of different phospholipid headgroups, showing a reduction in the amount of outer leaflet lipids and a significant increase in PS content. The breakdown of phospholipid species in terms of molar abundance for each species before and after the lipid exchange is shown individually for FIG. 4B) PC species, FIG. 4C) SM species, FIG. 4D) PE species, and FIG. 4E) PS species. Species with abundances smaller than 1% are summed together and presented as other lipids. Results are represented as mean±standard deviation of 3 different blood samples.

FIGS. 5A-5B: Abundance of FIG. 5A) PC and FIG. 5B) SM species in the outer leaflet of RBCs compared to the abundance of the same species in total lipid extract of RBCs. PC species with abundances smaller than 1% are summed together and presented as other PCs. Results are represented as mean±standard deviation of three different blood samples. SM nomenclature refers to the chain other than the sphingosine backbone, which is (18:1).

FIG. 6: Table showing the optimized conditions used for the lipid exchange process. The hemolysis values are presented as mean±standard deviation of at least 6 experiments.

### DETAILED DESCRIPTION OF THE INVENTION

It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below. Additionally, unless otherwise specifically noted, articles depicted in the drawings are not necessarily drawn to scale.

Throughout this disclosure, various publications, patents, and published patent specifications are/may be referenced by an identifying citation. Such disclosures of these publications, patents, and published patent specifications are hereby incorporated by reference into the present disclosure in their entirety to more fully describe the state of the art to which this invention pertains.

### Definitions

In order to facilitate review of the various embodiments of the disclosure, the following explanations of specific terms are provided:

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising” and the like.

As used herein, “therapeutic” is a generic term that includes both diagnosis and treatment. It will be appreciated that in these methods the “therapy” may be any therapy for treating a disease including, but not limited to, pharmaceutical compositions, gene therapy and biologic therapy such as the administering of antibodies and chemokines. Thus, the methods described herein may be used to evaluate a patient or subject before, during and after therapy, for example, to evaluate the reduction in disease state.

As used herein, “adjunctive therapy” is a treatment used in combination with a primary treatment to improve the effects of the primary treatment.

As used herein, “clinical outcome” refers to the health status of a patient following treatment for a disease or disorder or in the absence of treatment. Clinical outcomes include, but are not limited to, an increase in the length of time until death, a decrease in the length of time until death, an increase in the chance of survival, an increase in the risk of death, survival, disease-free survival, chronic disease, metastasis, advanced or aggressive disease, disease recurrence, death, and favorable or poor response to therapy.

As used herein, “decrease in survival” refers to a decrease in the length of time before death of a patient, or an increase in the risk of death for the patient.

As used herein, “patient” includes human and non-human animals. The preferred patient for treatment is a human. “Patient,” “individual” and “subject” are used interchangeably herein.

As used herein, “preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

As used herein, “poor prognosis” generally refers to a decrease in survival, or in other words, an increase in risk of death or a decrease in the time until death. Poor prognosis can also refer to an increase in severity of the disease.

As used herein, “screening” refers to the process used to evaluate and identify candidate patients that are affected by such disease/s.

As used herein, “diagnosing” refers to classifying a medical condition, predicting or prognosticating whether a particular abnormal condition will likely occur or will recur after treatment based on an indicia, detecting the occurrence of the disease in an individual, determining severity of such a disease, and monitoring disease progression.

As used herein, “individual” denotes a member of the mammalian species and includes humans, primates, mice and domestic animals such as cattle and sheep.

As used herein, “comprising, comprises and comprised of” are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

As used herein, “about” generally refers to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

As used herein, “and/or,” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

As used herein, “red blood cells” (RBCs) and stored red blood cells generally include RBCs present in whole blood, leukoreduced RBCs, platelet reduced RBCs, leukocyte and platelet reduced RBCs, and packed red blood cells (pRBCs).

As used herein, “RBC compositions” generally include, but are not limited to, any blood product comprising red blood cells (e.g., human blood), where the blood product provides, or is processed to provide, red blood cells suitable for use in humans, mammals, and/or vertebrates, such as for infusion.

As used herein, the term “poor quality” or “poor integrity” are used herein interchangeably to refer to an RBC storage sample that has reduced potency, has been compromised, would have potential adverse effects on recipients, or is not suitable for use in blood transfusions or research.

### General Description

MαCD is a cyclic molecule with a hydrophobic cavity that is large enough to form a complex with phospholipid tails but not with cholesterol. When in the proximity of red blood cells, MαCD, preloaded with lipids, exchanges the exogenous lipids with the lipids in the outer leaflet membrane. Thin layer chromatography of the supernatant after the exchange qualitatively showed the most abundant lipids in the outer leaflet membrane. Fluorescence microscopy confirmed the incorporation of fluorescent lipids into the membrane. Mass spectroscopy of the lipids in the supernatant after the exchange determined the types, composition, and saturation state of the phospholipids in the outer leaflet of the red blood cells.

### Examples

Certain embodiments of the present invention are defined in the Examples herein. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### Example 1

Reconstitution of the outer leaflet of senescent RBCs can decrease their in vitro clearance by macrophages:

Senescent RBCs lose their lipid membrane asymmetry and express phosphatidylserine (PS) on their outer leaflet.

The outer leaflet of RBCs stored in blood banking conditions for several months beyond their expiration dates were exchanged with 1:1 bSM:POPC molar ratio. The presence of PS in the outer leaflet exchanged or untreated RBCs was measured using FITC conjugated lactadherin. Lactdaherin binds to PS in a non Ca²⁺ dependent manner.

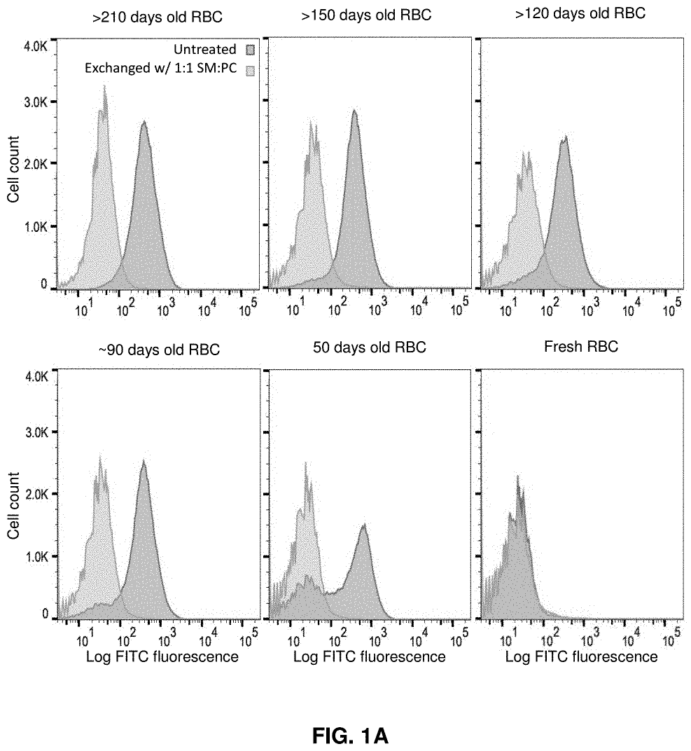

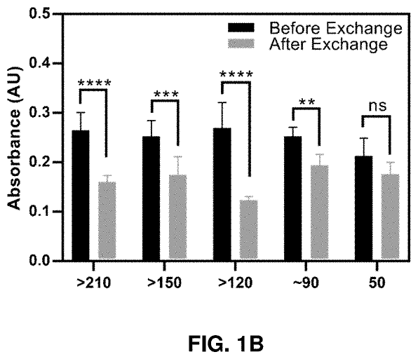

FIG. 1A shows the results of flow cytometry of exchanged or untreated RBCs of different age. After the exchange, the fluorescence level of all samples is reduced to that of fresh RBCs. Fresh RBCs do not express PS on their outer leaflet. Rather, the PS is completely removed from the outer leaflet of senescent cells after the exchange. As presence of PS on the outer leaflet of the membrane is believed to be a marker for macrophage clearance, the in vitro uptake of the RBCs by macrophages was measured.

RBCs of different age were incubated with THP-1 derived macrophages and the uptake was measured using a calorimetric method.

FIG. 1B depicts the uptake of exchanged or untreated senescent RBCs by macrophages. The calorimetric method is based on detecting the presence of hemoglobin in the macrophages and higher absorbance values indicate higher hemoglobin content.

The in vitro uptake of expired RBCs by macrophages is extremely reduced, after their outer leaflet is exchanged with 1:1 bSM:POPC.

The PS expression on the outer leaflet is dependent on the age of RBCs, as the two peaks obtained for 50 days old RBCs gradually merge into one. It is also observed that the PS expression level, regardless of age, can be lowered to that of fresh RBCs. PS is believed to constitute about 20% of the phospholipid composition of RBCs.

These results show that the exchange process is highly efficient in replacing all phospholipids in the outer leaflet, regardless of their abundance. As PS on the outer leaflet is known to be an eryptosis marker leading to macrophage clearance of senescent RBCs, the interaction of macrophage with senescent RBCs before and after the exchange were examined.

These results showed that phagocytosis of senescent RBCs was significantly decreased after exchange with 1:1 bSM:POPC. This can be attributed to the removal of PS from the outer leaflet, as macrophages utilize several receptors for recognition of PS to initiate the phagocytosis of RBCs.

These results also show that MαCD-mediated lipid exchange is useful for extending the shelf life of RBCs in the blood banks beyond their normal expiration dates.

Supporting Data

The Lipid Exchange Process is Optimized for Outer Leaflet Extraction:

The lipid exchange process was optimized to obtain a total extraction of the outer leaflet while the inner leaflet remains intact. The release of hemoglobin from the RBCs is considered as a sign of disrupting the cell membrane and consequently extracting the inner leaflet.

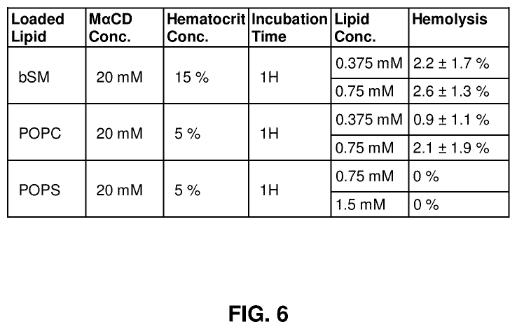

Therefore, the parameters involved in the lipid exchange process were varied to obtain lowest hemolysis. The hemolysis of RBCs during lipid exchange was measured using UV-Vis spectroscopy. The optimized conditions for MαCD mediated lipid exchange with RBCs and their respective hemolysis is shown in the Table in FIG. 6.

These optimized parameters were used in all further experiments. As the analysis of the extracted lipids required three different headgroups of exogenous lipids, brain SM (bSM), 1-palmitoyl-2-oleoyl-glycero-3-phosphocholine (POPC), and 1-palmitoyl-2-oleoyl-sn-glycero-3-phospho-L-serine (POPS), which showed the lowest hemolysis, were selected. Two MαCD concentrations were tested and although both concentrations were capable of mediating the exchange process, the smaller one was selected.

Confocal Microscopy Images Confirm that Lipid Exchange is Performed with the Outer Leaflet Only:

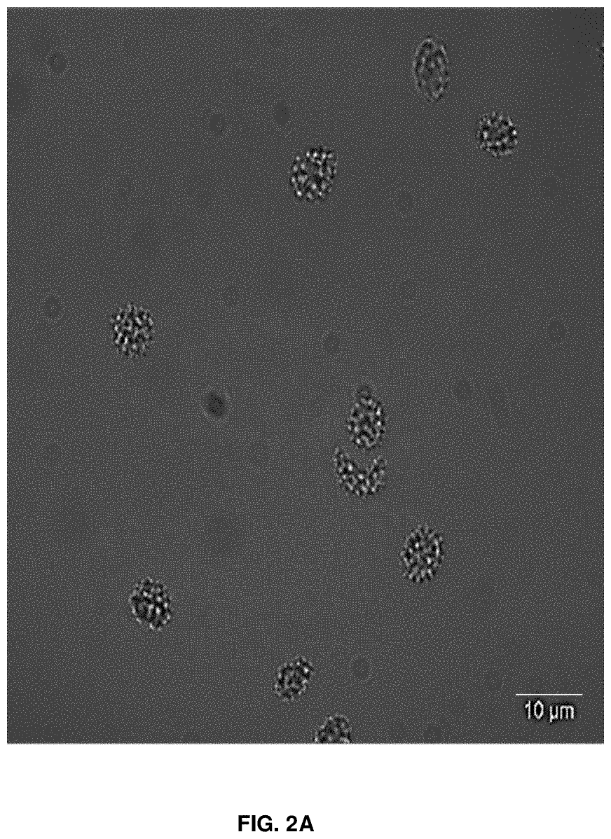

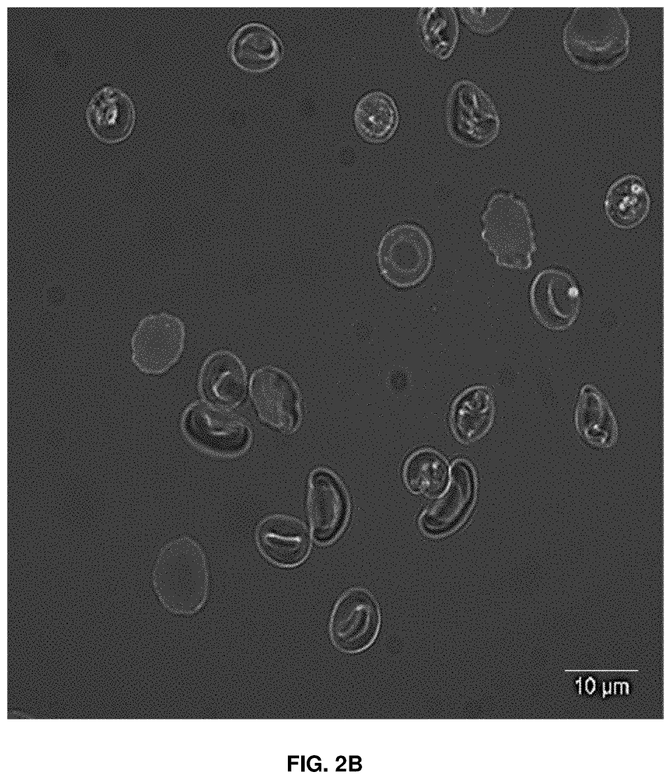

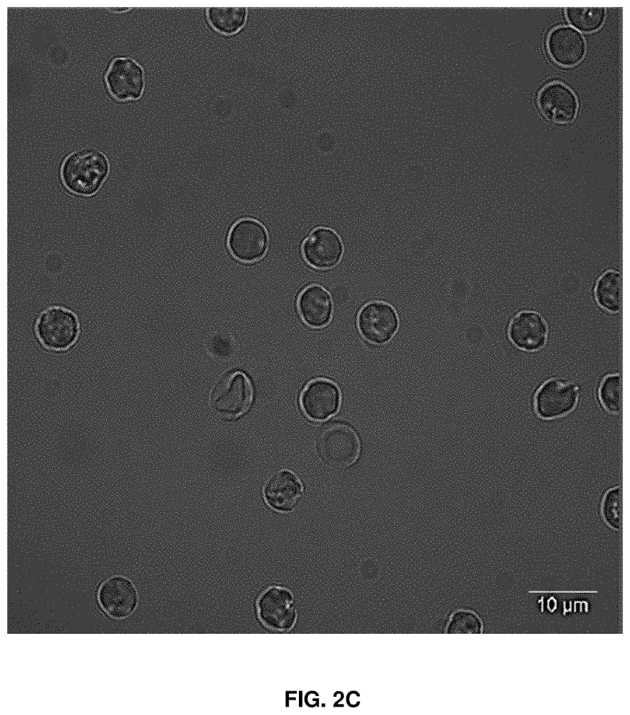

To confirm that MαCD mediates the lipid exchange and that the lipids are exchanged with the outer leaflet only, confocal microscopy was performed on the RBCs after the exchange. A mixture of POPS and 1,2-dipalmitoyl-sn-glycero-3-phosphoethanolamine-N-(7-nitro-2-1,3-benzoxadiazol-4-yl) (NBD-PE) with a molar ratio 19:1 POPS:NBD-PE was used. RBCs are incubated for one hour with the lipid mixture without MαCD (FIG. 2A) or with the lipid mixture loaded into MαCD (FIG. 2B).

The images in FIGS. 2A and 2B show that lipid exchange only occurs in the presence of MαCD as a mediator. The RBCs start to pick up some fluorescent lipid (NBD-PE) but compared to the MαCD loaded lipids, this is negligible.

In order to confirm whether the lipids are exchanged with the outer leaflet or not, sodium dithionite was used to quench NBD molecules. Sodium dithionite is relatively membrane impermeable and under short times cannot penetrate the cells. The NBD molecules are conjugated with the headgroup of the NBD-PE, and therefore are only available to the sodium dithionite if they are in the outer leaflet. FIG. 2C shows RBCs with an exchanged outer leaflet (with 19:1 POPS:NBD-PE molar ratio), after adding sodium dithionite, seconds before imaging. The fluorescence of the cells is diminished. While not wishing to be bound by theory, it is now believed that the fluorescent lipids were exchanged with the outer leaflet and that no fluorescent lipids are present in the inner leaflet.

Qualitative Analysis of the Lipids in the Supernatant of the Exchange Shows Removal of Lipids from the Outer Leaflet:

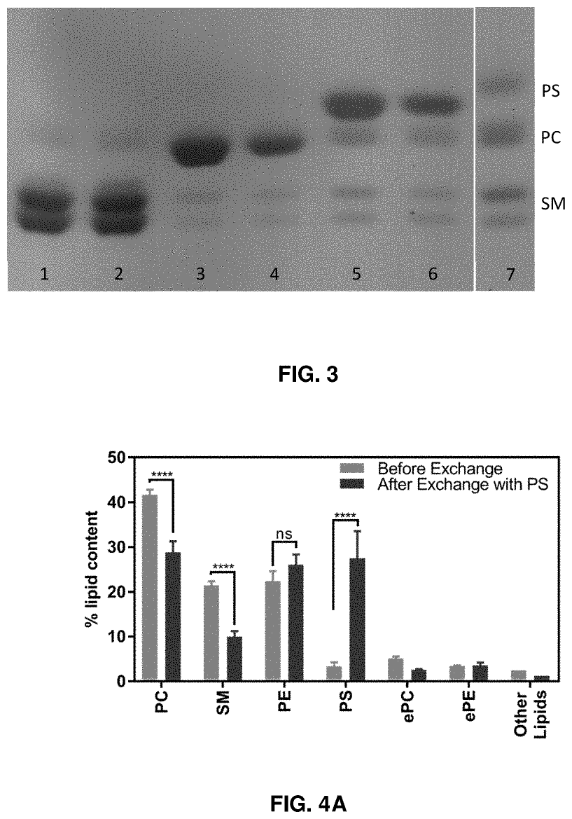

The exchange process was performed in the presence of excess amounts of phospholipids loaded into MαCD. After centrifuging the cells, the supernatant of the exchange included an abundance of the loaded lipid as well as the extracted lipids. Qualitative analysis of the lipids in the supernatant was performed using Thin Layer Chromatography (TLC). FIG. 3 shows the TLC plate for the experiments performed with the conditions shown in the Table in FIG. 6. When the exchange was performed with bSM loaded into MαCD (first and second lanes), dark bands for bSM and distinctive bands for PC were observed. The observed PC bands in the first two lanes show the extracted PC from the outer leaflet, while the extracted SM is masked under the dark band of the excess bSM in the supernatant. When POPC was the loaded lipid (third and fourth lanes), clear double bands was observed for the extracted SM from the outer leaflet, while the extracted PC is masked under the dark bands formed due to the excess POPC available. The absence of any bands representing PS in the first four lanes indicates that the exchange process is extracting only the outer leaflet and the inner leaflet is remained intact. When the exchange is performed with POPS loaded into MαCD (fifth and sixth lanes), double bands for the extracted SM and single bands for the extracted PC can be observed. As PS was not extracted, the PS bands only represent the excess POPS in the supernatant. It should be noted that although POPS was loaded with two concentrations, there are no changes in the extraction efficiency. Comparing ratio of intensity of the SM over PC bands in the fifth and sixth lanes, it is shown that the concentration of loaded lipid does not affect the extraction potency. The last lane shows total phospholipid composition of untreated RBCs as a control. Bands for SM, PC, and PS are also observed.

Quantitative Analysis of the Phospholipids in the RBCs after Exchange:

The total phospholipid composition of RBCs after exchange was obtained using mass spectrometry (MS). Exchange was performed with POPS, a phospholipid that exist in the RBCs only to trace amounts. As the exchange is with the outer leaflet only, comparing the phospholipid composition of untreated cells RBCs with cells after exchange showed what phospholipids reside in the outer leaflet. The choice of POPS was partially due to the fact that PS resides only in the inner leaflet of RBCs, therefore the POPS would not mask the lipids that reside in the outer leaflet.

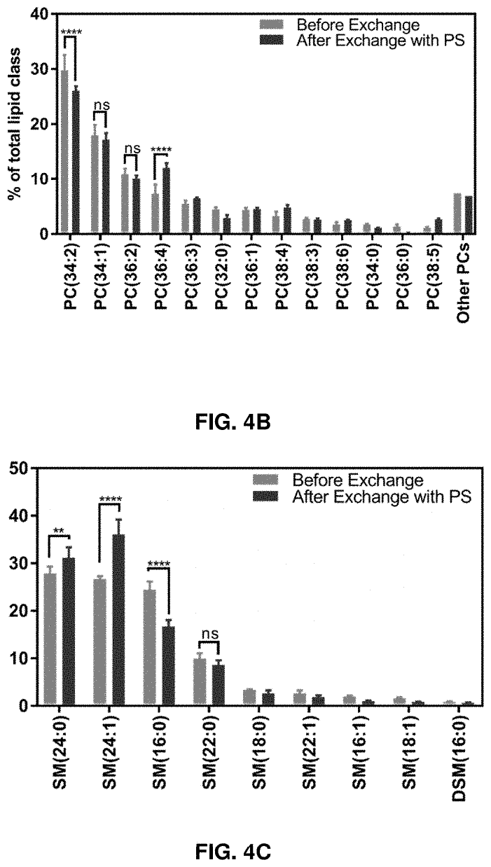

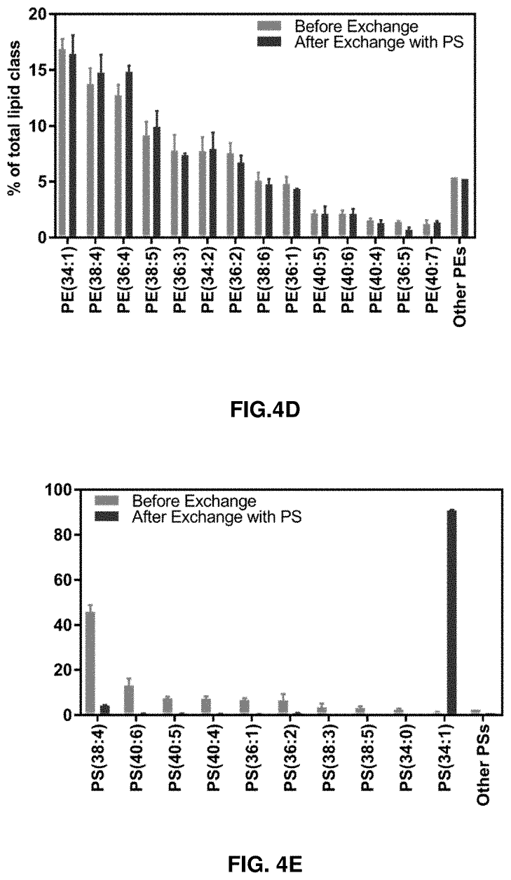

FIG. 4A shows the phospholipid content of RBCs after exchange compared to untreated cells. Drastic changes can be observed for the SM and PC groups, but the PE group shows no significant changes. This is attributed to the fact that the main constituents of the outer leaflet are SM and PC, while PE resides in the inner leaflet. The PS group also experiences a spike, which is an expected outcome, as the delivered lipid to the outer leaflet is POPS.

FIGS. 4B-4E show a breakdown of lipid species of each lipid headgroup for the exchanged RBCs compared to untreated cells. The lipid species are sorted from highest to lowest based on their content in the untreated cells. Of the PC species, PC 34:2 and PC 36:4 and of the SM species, SM 24:0, SM 24:1, and SM 16:0 experience significant changes after the exchange. PC 34:1 and PC 36:2 as well as SM 22:0 does not show any significant changes.

FIG. 4D shows that no species of PE has had a significant change after the exchange. When combined with the results of FIG. 4A, this shown that no species of PE exists in the outer leaflet.

The Phospholipid Composition of the Outer Leaflet is Obtained Using Mass Spectroscopy Analysis of the Lipids in the Supernatant:

The presence of excess exogenous lipids in the supernatant after the exchange, posed a problem in characterizing the extracted lipid. When analyzing the lipids in the supernatant using MS, the abundance of the loaded phospholipid will tend to mask a group of extracted lipids. Compared to the TLC plates where the bands are masked, here masking was observed as a spike in the molar percentage of the lipid species and headgroup. For instance, when POPC is the loaded lipid, the excess POPC will be characterized with the extracted PC species, masking the true values of the extracted PC species. The issue created a complication when bSM is the loaded lipid, as it consists of a wide range of different species of SM, eventually masking all species of SM that are extracted. To resolve this issue, for the samples where the loaded lipid is bSM, the values for SM were replaced by average values of SM from samples were the loaded lipid was POPC and POPS. The same analysis was performed for PC and PS as well. However, no extracted PS was observed in the samples where the loaded lipids were bSM or POPC. After averaging all values for each species, the composition of the phospholipids in the outer leaflet is obtained.

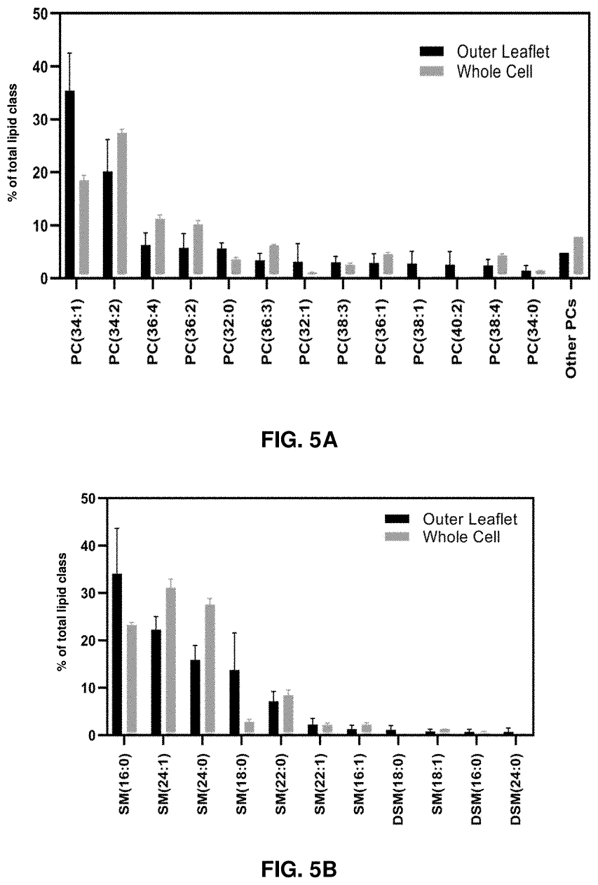

FIGS. 5A-5B show the breakdown of lipid species for two of the most abundant lipids in the outer leaflet of RBCs, PC and SM. It can be seen that PC 34:1 and PC 34:2 are the most abundant PC species while SM 16:0 and SM 24:1 are the most abundant SM species.

Methods:

Phospholipid Exchange Using Cyclodextrins:

Different amounts of various phospholipids were first dried out of their organic solvents using a SpeedVac. The dried lipids were then heated to a temperature above their melting point (usually 70° C.) and rehydrated with Dulbecco's Phosphate Buffered Saline (DPBS) at the same temperature. The mixture was then vortexed to form lipid vesicles. Methyl-α-cyclodextrin (MαCD) was resolved in DPBS at a final concentration of 80 mM. The turbid solution was centrifuged at 14,000×g for 5 minutes to obtain a clear solution.

This step removed the trapped air in the MαCD molecules and did not result in any precipitation. Desired volumes of the MαCD solution was added to the lipid vesicles suspension to obtain the final concentrations of MαCD and lipids. The solution was incubated at room temperature for 30 minutes and was then added to the triple washed RBCs to obtain the final hematocrit concentrations mentioned in the tables.

After one hour of incubation, the suspension was laid on 2 mL of 10% sucrose solution in DPBS and was centrifuged at 750×g for 5 minutes. The difference in the density helped precipitate the RBCs at the bottom of the tube and MαCD, excess exogenous lipids, as well as the extracted lipids were on top of the sucrose solution.

Measuring the Hemolysis of RBCs:

Presence of hemoglobin in the exchange solution is believed to be a disruption of the RBCs, lead to extracting the inner leaflet of the membrane. To monitor the amount of hemolysis after the exchange, the cells were centrifuged at 750×g for 5 minutes, 200 μL of the supernatant was collected, and its absorbance at 541 nm was measured using a plate reader. RBCs suspended in DPBS and water for 1 hour, at the same hematocrit concentrations as the ones used for exchange experiments, were used as negative and positive controls for the hemolysis. Both controls were also centrifuged at 750×g for 5 minutes.

Identification of Phospholipids in the Supernatant:

As phospholipid exchange proceeded, exogenous phospholipids were delivered to the membrane, replacing the endogenous phospholipids of the outer leaflet of RBCs. To identify the phospholipids in the supernatant of the exchange process, the top 1 mL of the supernatant was collected after centrifugation and the lipids were extracted using the Bligh & Dyer method.

According to the specific volumes provided by this method, solutions of 2:1 methanol:chloroform, chloroform, and water were added to the samples, with vigorous vortexing after each addition. The final milky suspension was centrifuged at 2000×g for 5 minutes and the bottom phase was collected using a Pasteur pipette, with positive pressure to avoid contamination. The collected solution was then dried using a SpeedVac and the dried lipids were then re-suspended in 100 μL of 1:1 methanol:chloroform solution. The latter was then carefully loaded to a high performance Thin Layer Chromatography (TLC) plate. The plate was then put in a TLC tank, containing the moving solution. The used moving solution 4:8:38:50 acetic acid:water:methanol:chloroform. When the solvent front reached the top of the plate, the plate was taken out of the tank and air dried. The plate was then sprayed with a solution of 3% cupric acetate in 8% phosphoric acid and air dried. Finally, the plate was charred in an oven at 185° C. for 5-10 minutes.

Confocal Microscopy of the RBCs after Exchange with a Fluorescent Lipid:

Delivering of the phospholipids to the membrane was studied using fluorescence. A quenching agent was used to diminish the fluorescence to examine whether the lipids were delivered to the outer or inner leaflet. Organic solutions of a fluorescent phospholipid (NBD-PE) and POPS were mixed to a molar ratio of 1:19 NBD-PE:POPS. The other steps prior to the exchange experiment were as mentioned before. After the exchange, the cells were centrifuged at 750×g for 5 minutes, the precipitated RBCs were collected, re-suspended in 10% sucrose in DPBS, loaded onto a glass slide, and imaged using a confocal microscope. Sodium dithionite solution was made to a concentration of 50 mM and was added to an aliquot of the exchanged RBCs 1 minute prior to loading onto glass slides and imaging.

Mass Spectrometry Analysis of the Lipids:

Quantification of the phospholipids and their respective species were performed using mass spectrometry. After exchanging the outer leaflet of RBCs with POPS, the cells were collected, lysed using water to remove the hemoglobin, and a total lipid extraction was performed on the cells using the Bligh & Dyer method. The lipids in the supernatant of the exchange process, after separate exchanges with bSM, POPC, and POPS were also extracted using the Bligh & Dyer method. The lipids were then dried, weighed, and sent for analysis to Kansas Lipidomics Research Center. The technique for lipidomics was Liquid Chromatography followed by Mass Spectrometry (LC-MS).

Analysis of the Lipidomics Results:

The lipidomics resulted in a long sheet of the main phospholipid groups, lyso groups, and their naturally occurring species in mammalian cells. The only changes made in the data were removing the entries that were below the detection limit of the instrument. After that, the species of each phospholipid headgroup were summed together to obtain the total abundance of each headgroup. Two-way ANOVA was performed on the results to obtain the significance of changes compared to the untreated RBCs as control.

Detection of PS in the Outer Leaflet Before and After Exchange:

Presence of PS in the outer leaflet of the mammalian cells was attributed to the apoptosis of the cells. Milk fat globule epidermal growth factor 8 protein (AKA lactadherin) conjugated with FITC was used to detect the PS in the outer leaflet of red bool cells. Senescent RBCs (aged more than 60 days) were washed three times, brought to 1% hematocrit content, and were then incubated with 20 μmol lactadherin/FITC for 30 minutes. The outer leaflet of the RBCs from the same donors were exchanged with 1:1 bSM:POPC, and the cells were incubated with lactadherin/FITC as described. A flow cytometer was used to detect the FITC intensity for 100,000 events.

Macrophage Uptake of Senescent RBCs:

The macrophages cleared the apoptotic RBCs from circulation through different pathways, one of which was initiated with detection of PS on the outer leaflet. Macrophages were derived from THP-1 monocytes. Briefly, the THP-1 cells were incubated for 48 hours prior to use with 20 ng/mL of Phorbol 12-Myristate 13-Acetate (PMA), in a 96 well plate. The macrophages were washed twice with warm media, then incubated with exchanged and untreated senescent RBCs (separately) at a ratio of 1:20 macrophages:RBCs for two hours.

The uptake of RBCs was measured using a calorimetric method based on specific oxidation of 2,7-diaminofluorene (DAF) by hemoglobin. The macrophages were washed two times with media to remove the excess RBCs and then the surface bound RBCs were lysed using a hypotonic solution (0.25% NaCl) for 3 minutes. The wells were washed twice again using media to remove any RBCs or free hemoglobin in the well. The remaining cells were then lysed using a solution of 0.2M Tris-HCl in 6M urea. A solution of 100 mg DAF in 10 mL of a 90% glacial acetic acid in water was then made (DAF stock). 1 mL of DAF stock was added to 10 mL of 0.2M Tris-HCl in 6M urea solution and 1004, of 30% hydrogen peroxide was added. 1004, of the final product was added to each well, and after 5 minutes of incubation the absorbance of each well was measured at 620 nm.

### Example 2

Applications

According to the NIH, RBCs for transfusion are to be stored refrigerated in a preservative solution, which extends their shelf-life. Generally, these preservative solutions include saline-adenine-glucose-mannitol (SAG-M) which enable refrigerated storage of RBCs for up to 42 days following collection. This expiry is based on criteria set by the United States of America Food and Drug Administration, which requires that 75 percent of transfused RBCs must be recoverable in the peripheral blood circulation 24 h after transfusion.

One protocol for the storage of red blood cells (for up to 42 days) is the collection of blood into anticoagulant solutions (such as citrate-dextrose-phosphate). Red cell concentrates are prepared by the removal of plasma and, in some cases, also leukocytes. The product is stored at 4±2° C. in a slightly hypertonic additive solution, generally SAG-M (sodium, adenine, glucose, mannitol).

Generally, blood storage methods include acid citrate dextrose (ACD), citrate-phosphate-dextrose solution (CPD) and citrate-phosphate-dextrose-adenine solution (CPDA) or a combination thereof. Citrate or other anticoagulants are useful to prevent clotting. An energy source such as dextrose may be added to maintain blood's metabolic functions even at refrigerated temperatures. In certain embodiments, phosphate ions may be used to buffer the lactate produced from dextrose utilization. In certain embodiments, the anticoagulant is hirudin, heparin, coumarin, warfarin, dicumarol, aspirin or combinations thereof.

It is to be understood that the present disclosure contemplates the use of “RBC compositions” which include, for example, whole blood collected red blood cells, collected red blood cells and red blood cell concentrates, such as packed red blood cells (pRBCs; e.g., red blood cells with increased hematocrit and/or not containing additive solution).

Such RBC compositions may include chemicals, such as pathogen-inactivating compounds and other components, such as buffers and other solutions, such as red blood cell additive solutions (e.g., any solution described in herein, such as SAG-M), including salts or buffered solutions, anticoagulants, and the like).

In some embodiments, RBC compositions for use as disclosed herein (e.g., input RBCs, prior to subjecting to treatment, prior to mixing with a pathogen-inactivating compound) may comprise plasma and/or anticoagulant, with and/or without additive solutions. Anticoagulants and useful volumes thereof are well known in the art.

In some embodiments, the red blood cell compositions described herein have been leukoreduced prior to use in the methods (e.g., methods of treating and/or methods of preparing) described herein.

In some embodiments, the red blood cell compositions have not been leukoreduced.

Any red blood cell composition that will come into contact with, or be introduced into, a living human, mammal, or vertebrate, where such contact carries a risk of transmitting disease due to contaminating pathogens may be treated as disclosed herein.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

What is claimed is:

1. A method for extending the shelf life of donor blood containing red blood cells (RBC), comprising:
i) obtaining, or having obtained, a supply of donor blood and/or RBC from a subject;
ii) exposing the donor blood and/or RBC to a quantity of at least one cyclodextrin composition, wherein the cyclodextrin composition comprises at least one cyclodextrin complexed with at least one exogenous lipid, in an amount sufficient to mediate the exchange of the exogenous lipids with at least one type of lipid present on the outer membrane of the RBC; and,
iii) extending the shelf life of the donor blood and/or RBC of step ii).

2. The method of claim 1, further including the step of: iv) preserving the cyclodextrin-treated donor blood and/or RBC of step iii) in storage.

3. The method of claim 1, wherein the cyclodextrin comprises methyl-α-cyclodextrin (MαCD).

4. The method of claim 1, wherein the exogenous lipids comprise one or more of: phosphatidylcholines (PC) and sphingomyelins (SM).

5. The method of claim 1, wherein the donor blood and/or RBCs are exposed to the cyclodextrin composition for a period of time sufficient to reconstitute the lipids in the outer leaflet of the membranes of the RBC in the donor blood.

6. The method of claim 5, wherein the amount of time of the cyclodextrin composition exposure is determined by measuring the hematocrit levels in the donor blood before and after the cyclodextrin composition treatment of the donor blood.

7. The method of claim 6, wherein the amount of time of the cyclodextrin composition exposure ranges from about 15 minutes to about 2 hours, from about 30 minutes to about hour, or about 1 hour.

8. The method of claim 1, wherein the cyclodextrin composition-treated RBC have a shelf life of at least 90 days; at least 120 days; at least 150 days; or, at least 210 days.

9. The method of claim 1, wherein the blood is mammalian blood, and/or the RBC are from mammalian blood.

10. The method of claim 1, wherein the blood is human blood, and/or the RBC are from human blood.

11. The method of claim 1, wherein the cyclodextrin composition is added at a concentration of between 10 mM and 20 mM.

12. The method of claim 1, wherein the mediation is determined by measuring the membrane integrity of the cyclodextrin composition-treated RBC as compared to non-cyclodextrin composition-treated RBC.

13. A kit intended for the treatment of red blood cells (RBC) in donor blood, the kit comprising:
a quantity of a cyclodextrin composition, wherein the cyclodextrin composition comprises at least one cyclodextrin complexed with at least one exogenous lipid; and,
instructions for treating the RBC in donor blood.

14. The kit according to claim 13, wherein the cyclodextrin comprises methyl-α-cyclodextrin (MαCD).

15. The kit of claim 13, wherein the exogenous lipids comprise one or more of: phosphatidylcholines (PC) and sphingomyelins (SM).

16. A unit of donor blood and/or red blood cells (RBC) suitable for infusion into a subject, comprising a supply of cyclodextrin-treated RBC having a quantity of exogenous lipids present on the outer membranes of the RBC, wherein the exogenous lipids are complexed with a cyclodextrin.

17. The kit according to claim 16, wherein the cyclodextrin comprises methyl-α-cyclodextrin (MαCD).

18. The kit of claim 16, wherein the exogenous lipids comprise one or more of: phosphatidylcholines (PC) and sphingomyelins (SM).

19. A method of infusing donor blood and/or red blood cells (RBC) into a subject in need thereof, comprising infusing into the subject a quantity of cyclodextrin-treated RBC having a quantity of exogenous lipids present on the outer membranes of the RBC, wherein the exogenous lipids are complexed with a cyclodextrin.

20. The method according to claim 19, wherein the cyclodextrin comprises methyl-α-cyclodextrin (MαCD).

21. The method of claim 19, wherein the exogenous lipids comprise one or more of: phosphatidylcholines (PC) and sphingomyelins (SM).

22. A red blood cell composition comprising cyclodextrin-treated red blood cells (RBC) having a quantity of RBC having a quantity of exogenous lipids present on the outer membranes of the RBC, wherein the exogenous lipids are complexed with a cyclodextrin; and optionally,
one or more of anticoagulants, pathogen-inactivating compounds, and other RBC additive solutions.

23. The red blood cell composition of claim 22, wherein the cyclodextrin comprises methyl-α-cyclodextrin (MαCD).

24. The red blood cell composition of claim 22, wherein the exogenous lipids comprise one or more of: phosphatidylcholines (PC) and sphingomyelins (SM).

25. A method for decreasing morbidity of a blood transfusion recipient, comprising:
treating the blood transfusion recipient with an effective amount of a cyclodextrin-treated red blood cells (RBC) having a quantity of RBC having a quantity of exogenous lipids present on the outer membranes of the RBC, wherein the exogenous lipids are complexed with a cyclodextrin; and,
decreasing the morbidity of the blood transfusion recipient.

26. The method of claim 25, wherein the cyclodextrin comprises methyl-α-cyclodextrin (MαCD).

27. The method of claim 25, wherein the exogenous lipids comprise one or more of: phosphatidylcholines (PC) and sphingomyelins (SM).
